(19) United States
(12) Patent Application Publication
(10) Pub. No.: US 2025/0073158 A1
(43) Pub. Date: Mar. 6, 2025

(54) Active substance comprising galactomannans from medicago sativa and cosmetic uses thereof

(71) Applicant: SOCIETE INDUSTRIELLE LIMOUSINE D'APPLICATION BIOLOGIQUE, SAINT VIANCE (FR)

(72) Inventor: Jean PAUFIQUE, OBJAT (FR)

(21) Appl. No.: 18/822,670

(22) Filed: Sep. 3, 2024

(30) Foreign Application Priority Data
Sep. 4, 2023 (FR) FR2309261

(51) Int. Cl. A61K 8/9789; A61K 8/73; A61Q 19/00; A61Q 19/08

(52) U.S. Cl. CPC A61K 8/9789; A61K 8/737; A61Q 19/007; A61Q 19/08

(57) ABSTRACT

The present invention relates to a cosmetic active substance comprising galactomannans obtained from Medicago sativa seeds and its cosmetic uses. This active substance has a complexion-perfecting effect, and is mixed and multi-ethnic, designed for all types of healthy skin: Caucasian, Asian, African-American and Hispanic-American, whatever their age.

### TECHNICAL FIELD

The invention relates to a cosmetic active substance comprising galactomannans obtained from Medicago sativa seeds and its cosmetic uses. This active substance advantageously has a mixed and multi-ethnic complexion-perfecting effect, that is it is intended for all types of healthy skin, whether Caucasian, Asian, African-American or Hispanic-American.

### PRIOR ART

Over time, standards of ideal beauty in society have kept changing. However, despite cultural differences, the cosmetics industry has long upheld Western standards in promoting its products. Although make-up shade variations were first introduced in the 70s in the USA, opening the doors to a more inclusive beauty world, it was only with the rise of social media and its influencers from all corners of the globe that the movement really took off. Driven by the emergence of diversity, equity, and inclusion, a re-prioritization of individual needs has gradually shaped a new face of beauty where each individual feels neither excluded nor marginalized. These principles go hand in hand with the growing demand for ultra-personalized care, driven by all age groups.

This approach now extends beyond skin color to encompass the specific characteristics of each ethnic group, gender, or stage of life. The study of these structural and functional specificities of the skin has aroused great interest in dermatology, and enabled the cosmetics industry to extol the virtues of “pluralistic beauty”. Whether male, female, young, old, and no matter where in the world they originate, all skin types have their own particularities and issues.

Nevertheless, men and women share common expectations in terms of moisturizing and anti-aging skincare, including complexion-perfecting skincare. A radiant, even complexion is therefore a universal concern, regardless of ethnicity or gender. Thus, despite differences in needs and concerns according to ethnicity, age and gender, three major areas of beauty are emerging to address the universal expectations of skincare consumers: skin quality, complexion and anti-aging.

For these reasons, there is a need for a cosmetic active substance to address the concerns and expectations of mixed and multi-ethnic beauty.

To meet this need, the inventor focused on the remarkable properties of alfalfa, already known for its beneficial effects on Caucasian skin, to develop a new naturally-derived ingredient that meets the expectations of mixed and multi-ethnic beauty.

Thus, the inventor has developed a new cosmetic active substance, namely an extract of Medicago sativa, enriched with galactomannans to provide mixed and multi-ethnic cosmetic benefits, in particular a complexion-perfecting effect for all healthy skin types, whether Caucasian, Asian, African-American or Hispanic-American.

### SUMMARY OF THE INVENTION

The invention thus relates to the cosmetic use of a cosmetic active substance comprising at least one hydrolysate comprising galactomannans obtained from Medicago sativa seeds, for its complexion-perfecting effect on healthy skin, preferentially healthy Caucasian and Asian and African-American and Hispanic-American skin.

According to a preferred object, the invention relates to the cosmetic use of said cosmetic active substance for its complexion-perfecting and moisturizing effects, as well as its anti-aging properties. These cosmetic effects, combined in a single cosmetic active substance, are of particular interest in meeting the needs of mixed and multi-ethnic beauty.

In this context, the inventor evaluated the mixed and multi-ethnic cosmetic benefits of this cosmetic active substance, in particular the complexion-perfecting effect for several skin types, namely Caucasian and Asian and African-American and Hispanic-American skins. The active substance's performance has been demonstrated from cell to volunteer. Firstly, pro-collagen activity was measured on fibroblasts from female and male volunteers of different ages and ethnicities. The cosmetic active substance according to the invention has also demonstrated its complexion-perfecting power, that is an improvement in complexion radiance as well as complexion evenness, in particular a reduction in pigmentary disorders, on all skin types, meeting consumer expectations.

According to another aspect, the invention is also aimed at a new cosmetic active substance comprising at least one extract obtained from Medicago sativa seeds, wherein the Medicago sativa extract comprises, by weight of the total weight of the extract, at least 35% sugars and at most 25% proteins.

According to a preferred object of the invention, the sugar fraction of the extract comprises at least 60% galactomannans by weight of the total weight of sugars in the extract, the extract comprising at least 60% galactomannans in the form of oligo-galactomannans or poly-galactomannans, with molecular weights between 360 Da and 1260 kDa, by weight of the total weight of sugars in the extract, resulting in improved cosmetic benefits for each consumer.

More preferentially, the extract according to the invention comprises at least 30% galactomannans with molecular weights between 3.6 kDa (DP20) and 1260 kDa (DP7000), by weight of the total weight of sugars in the extract.

The cosmetic active substance comprising Medicago sativa extract can take a variety of forms: in liquid, solid or film form, preferentially in liquid form. This advantageously comprises at least one excipient chosen from a preservative, an antioxidant, a stabilizer, an atomization medium, and/or a combination thereof.

According to another particularly preferred object, the Medicago sativa extract is a Medicago sativa hydrolysate, more preferentially an enzymatic hydrolysate.

The active substance according to the invention can be obtained by any suitable method. According to a particularly attractive embodiment, the active substance according to the invention is obtained by a method comprising the following steps:
a. Solubilization of Medicago sativa seeds in water at a rate of at least 50 g/L,
b. Extraction by hydrolysis, preferentially enzymatic hydrolysis,
c. Separation of the soluble and insoluble phases,
d. Heat treatment,
e. Concentration of the soluble phase,
f. Purification by ion chromatography,
g. Potentially, filtration and sterilizing filtration.

Thus, the invention also relates to the method for extracting and preparing the active substance described above.

On the other hand, the invention also relates to a cosmetic composition comprising at least 0.1% of a cosmetic active substance according to any of the preceding objects, by weight of the total weight of the composition.

Finally, the invention relates more advantageously to the use of this cosmetic active substance according to the invention or of the composition comprising it, intended for a cosmetic treatment for men and women, by topical application to the skin, preferentially to healthy skin of the Caucasian, Asian, African-American, but also Hispanic-American type for multiple cosmetic benefits, namely a complexion-perfecting, moisturizing and anti-aging effect, regardless of the skin type.

The inventor has also developed a natural, mixed and multi-ethnic active substance that meets universal cosmetic expectations in terms of inclusive beauty.

Other features and advantages will emerge from the detailed description of the invention, examples and figures that follows.

### BRIEF DESCRIPTION OF THE FIGURES

FIG. 1 shows the effect of the active substance according to the invention, formulated as a 2.5% emulsion, on cell renewal in young Caucasian volunteers, compared with a placebo formula.

FIG. 2 shows the effect of the active substance according to the invention, formulated as a 2.5% emulsion, on cell renewal in mature Caucasian volunteers, compared with a placebo formula.

FIG. 3 shows the effect of the active substance according to the invention, formulated as a 2.5% emulsion, on the quality of dermal matrix fibers in mature Caucasian volunteers after 42 days of twice-daily treatment.

FIG. 4 shows the effect of the active substance according to the invention, formulated as a 2.5% emulsion, on the radiance parameter of the complexion of young and mature panels, 15 minutes after a single application and after 7, 21 or 42 days of treatment.

### DETAILED DESCRIPTION OF THE INVENTION

### Definition

The terms “inclusive beauty”, “universal beauty” or “mixed and multi-ethnic beauty” within the meaning of the present invention, mean an improvement in skin quality, complexion radiance, skin tone homogeneity and skin hydration, regardless of whether the healthy skin is Caucasian, Asian, African-American or Hispanic-American, on either men or women and regardless of their age.

The term “Medicago sativa extract”, within the meaning of the present invention, means any molecule or mixture of at least two molecules obtained from a raw material, namely alfalfa, that is the species Medicago sativa, regardless of the method of extraction of said molecule or molecules. It may for example be an extract obtained by aqueous and/or hydroalcoholic and/or hydro glycolic extraction and/or obtained after at least one hydrolysis step, for example enzymatic or acid hydrolysis.

The term “Medicago sativa hydrolysate”, within the meaning of the invention, means any extract derived from the plant Medicago sativa, obtained by a method comprising at least one step of hydrolysis of Medicago sativa. The term Medicago sativa excludes molecules produced solely by fermentation of Medicago sativa by a microorganism or an infusion/decoction/maceration of Medicago sativa. Thus, within the meaning of the invention, such an extract is a naturally-derived ingredient obtained by specific and defined chemical and/or biological methods aimed at chemically modifying it; such modifications are intentional, and make it possible to obtain molecule structures which are therefore no longer identical to the molecules present in nature.

The term “cosmetic active substance”, within the meaning of the invention, means an extract comprising at least one molecule, preferentially a set of molecules, with a cosmetic effect, particularly when applied topically, the cosmetic effect in particular being a complexion-perfecting effect.

The term “complexion-perfecting” within the meaning of the invention, means an improvement in complexion radiance and a reduction in post-inflammation hyperpigmentation marks on the skin.

### Active Substance According to the Invention

The present invention therefore relates to a cosmetic active substance comprising at least one extract obtained from Medicago sativa seeds, wherein the Medicago sativa extract comprises, by weight of the total weight of the extract, at least 35% sugars and at most 25% proteins.

Medicago sativa, also known as alfalfa or lucerne, is a forage plant native to Asia that has been cultivated throughout the world, notably for its nutritional qualities and medicinal properties. It is adorned with clusters of flowers in various shades of purple, which then develop into spiraling pod-shaped fruits containing the seeds. Furthermore, the roots harbor microorganisms that establish a nitrogen-fixing symbiosis, helping both plant development and soil enrichment. The cultivation of alfalfa is therefore of great ecological importance for the environment and biodiversity, in addition to its nutritional and medicinal properties.

Access (as defined by the Nagoya Protocol) to the raw plant material (Medicago sativa) was carried out in accordance with the national ABS (Access and Benefit-Sharing) regulations of the country of origin, France.

Alfalfa seeds are naturally rich in polysaccharides such as mannose and galactose polymers, which the inventor has remarkably exploited to obtain an extract enriched in galactomannans of interest.

The active substance according to the invention comprising the Medicago sativa extract is preferentially composed mainly of galactomannans, more preferentially the extract comprises at least 60% galactomannans, by weight of the total weight of sugars comprised in the extract. This composition was determined by ion chromatography.

The inventor has characterized the sugars present in the extract according to the invention by chromatography (LC-CAD). The total sugar content, meanwhile, can be determined by the DUBOIS method (Dubois M. et al., Analytical chemistry, 28, 3, 350-356, 1956).

According to a preferred object, the extract according to the invention comprises at least 60% sugars with molecular weights between 360 Da and 1260 kDa (DP2 to DP7000), by weight of the total weight of sugars in the extract.

According to a further preferred object of the invention, the extract according to the invention comprises at least 30% sugars with molecular weights between 3.6 Da (DP20) and 1260 kDa (DP7000), by weight of the total weight of sugars in the extract.

Very preferentially, the extract according to the invention comprises at least 60% sugars with molecular masses between 360 Da and 1260 kDa (DP2 and DP7000), by weight of the total weight of sugars in the extract, and at least 30% of sugars with molecular masses between 3.6 kDa (DP20) and 1260 kDa (DP7000), by weight of the total weight of sugars in the extract.

Finally, according to a further object, the extract comprises at most 25% sugars with molecular masses less than or equal to 180 Da (DP1), by weight of the total weight of sugars in the extract, preferentially at most 20% of sugars with molecular masses less than or equal to 180 Da.

The composition of the sugars making up the extract according to the invention has also been determined, in particular by ion chromatography. The extract advantageously comprises galactose, mannose, glucose and fructose. Very advantageously, it comprises predominantly galactose and mannose.

The extract according to the invention is also rich in galactomannans. To improve and optimize this enrichment, the extract is very preferentially obtained using hydrolysis, even more preferentially at least enzymatic hydrolysis. Thus, the Medicago sativa extract according to the invention is preferentially a hydrolysate, more preferentially an enzymatic hydrolysate.

The extract according to the invention can be obtained by any extraction method. However, in a particularly preferred manner, the extract is likely to be obtained by a method comprising at least the following steps:
a. Solubilization of Medicago sativa seeds in water at a rate of at least 50 g/L,
b. Extraction by hydrolysis, preferentially enzymatic hydrolysis,
c. Separation of the soluble and insoluble phases,
d. Heat treatment,
e. Concentration of the soluble phase,
f. Purification by ion chromatography,
g. Potentially, filtration and sterilizing filtration.

The cosmetic active substance according to the invention may also advantageously comprise at least one excipient chosen from a preservative, an antioxidant, a stabilizer, an atomization medium, and/or their combination.

The extract according to the invention present in the cosmetic active substance can then be in liquid form or in solid or film form.

When it is in liquid form, the active substance according to the invention is exclusively composed of Medicago sativa extract accompanied by stabilizers and/or storage systems.

The extract in liquid form is preferentially in the form of a clear liquid aqueous solution, with a low odor and a very light-yellow to yellow color.

When it is in liquid form, the content of dry matter of the extract can be determined by weighing the residues resulting from the drying of the extract according to the invention at 105° C. in an oven until a constant weight is obtained. Preferentially, the extract according to the invention in liquid form has a dry matter content of 20 g/L to 70 g/L, preferentially 20 to 55 g/L, even more preferentially 22 g/L to 32 g/L.

When it is in solid form, the active substance according to the invention is preferentially composed of Medicago sativa as previously described and by a support selected from maltodextrin, gum arabic, or soy lecithin. According to one particularly suitable embodiment, the extract represents at least 10% by weight of the active substance and the support at most 90% by weight of the active substance.

In the case of a solid form wherein the active substance is associated with a support, the protein contents, sugars and galactomannans relative to the dry matter in the active substance can be modified, because the supports (maltodextrin, gum arabic) are exclusively saccharides.

The active substance according to the invention can also be presented in the form of a film, as described in patent FR3079145. In this case, the extract of Medicago sativa preferentially represents at least 0.1% by weight of the film.

When it is in the form of a film, the active substance comprises:
at least the extract of Medicago sativa according to the invention.
at least one mineral filler, and
at least one polymer of natural origin, and
at least one plasticizer, and
at least one surfactant.

The polymer of natural origin may be chosen from: Pectin, tamarind gum, alginate, pullulan, psyllium, xanthan, guar, tara, carob, agar, gum arabic, gellan, dextran, carrageenan, cellulose, konjac and chitosan.

The plasticizer may be chosen from: Glycerol, sorbitol, saccharose, erythritol, urea, propylene glycol and butylene glycol.

The mineral filler may be chosen from: Calcium carbonate, green clay, kaolin, perlite, talc, magnesium silicate, mica, diatomaceous sericite, silica, calcium sulfate, calcium chloride, potassium chloride, iron oxide and zinc oxide.

The active substance may further comprise a pigment for coloring the film.

### Composition According to the Invention

The purpose of the active substance according to the invention is to be integrated into a composition, in particular a composition comprising at least 0.1% by weight of said active substance and a physiologically acceptable medium, preferentially a cosmetically acceptable medium.

These compositions may in particular be in the form of oil-in-water emulsions, water-in-oil emulsions, multiple emulsions (Water/Oil/Water or Oil/Water/Oil) which may optionally be microemulsions or nanoemulsions, or in the form of solutions, suspensions, hydrodispersions, aqueous gels, powders, or foundation or in the form of a film. They may be more or less fluid and have the appearance of creams, emulsions, gels, masks or any other aspects known to the person skilled in the art.

Preferentially, these can be compositions comprising between 0.1 and 20% of the active substance according to the invention, notably in liquid form, more preferentially between 0.5 and 10%.

These compositions comprise, in addition to the active substance, a physiologically acceptable medium such as a cosmetically acceptable medium, that is to say which does not cause sensations of discomfort for the user, such as redness, tugging or tingling.

As an ingredient, the compositions according to the invention may also contain at least one compound selected from:
oils, which may be chosen in particular from linear or cyclic, volatile or non-volatile silicone oils,
waxes, such as ozokerite, polyethylene wax, beeswax or carnauba wax,
silicone elastomers,
surfactants, preferably emulsifiers, whether non-ionic, anionic, cationic or amphoteric,
co-surfactants, such as linear fatty alcohols,
thickeners and/or gelling agents,
humectants, such as polyols such as glycerin,
dyes, preservatives, fillers,
tighteners,
sequestrants,
perfumes,
and mixtures thereof, without this list being exhaustive.

Examples of such ingredients are cited in particular in the CTFA

Dictionary (International Cosmetic Ingredient Dictionary and Handbook published by the Personal Care Product Council).

Of course, a person skilled in the art will take care to choose any additional, active or non-active compounds, and their quantity, such that the advantageous properties of the mixture are not, or substantially not, altered by the envisaged additive.

### Method for Preparing the Extract According to the Invention

The extract constituting or contained in the active substance according to the invention can be obtained by any means.

However, according to a preferred embodiment, the extraction method comprises at least one step of extraction from Medicago sativa, such as at least one hydrolysis, preferentially at least one enzymatic hydrolysis, more preferentially at least one enzymatic hydrolysis using a carbohydrase.

According to a particularly suitable embodiment, the active substance according to the invention is obtained by implementing the following steps:
a. Solubilization of Medicago sativa seeds in water at a rate of at least 50 g/L,
b. Extraction by hydrolysis, preferentially enzymatic hydrolysis,
c. Separation of the soluble and insoluble phases,
d. Heat treatment,
e. Concentration of the soluble phase containing galactomannans,
f. Purification by ion chromatography for galactomannan enrichment,
g. Potentially, filtration and sterilizing filtration.

The heat treatment step is advantageously carried out to inactivate the enzyme(s) present. This inactivation is then carried out according to the recommendations of the enzyme supplier(s).

The separation of the soluble and insoluble phase is carried out by any means known to a person skilled in the art, for example by centrifugation, filtration or decantation. The soluble and insoluble phases are separated to recover the soluble phase containing galactomannans.

The extract obtained at this stage may be further concentrated. Preferentially, the extract is purified, preferentially by successive ultrafiltration steps through membranes of different porosity, while retaining the galactomannans at each step and/or by a chromatographic method. In the context of the invention, the extract obtained after the soluble phase concentration step undergoes at least one purification step to enrich the galactomannan content, preferentially by ion chromatography.

The extract obtained after hydrolysis and concentration is an extract of Medicago sativa, and constitutes a first form of the active substance according to the invention, being in liquid form at that time.

Another form of the active substance according to the invention is obtained after hydrolysis, concentration and purification, and is also in liquid form.

The extract obtained can then be dried and associated or not with a support, in order to be in solid form. This phase can be carried out by implementing the following steps:
an atomization support, preferably maltodextrin, is added to the extract of Medicago sativa, at most 90% (by mass/volume);
this solution is then concentrated under vacuum;
an antimicrobial treatment can be applied;
atomization makes it possible to obtain a powder.

The steps of the methods described above, taken individually are common in the field of extraction of active substances from natural raw materials and the person skilled in the art is able to adjust the reaction parameters on the basis of his general knowledge.

### Cosmetic Use

According to one aspect, the invention targets the cosmetic use of a cosmetic active substance comprising at least one hydrolysate comprising galactomannans obtained from Medicago sativa seeds, for its complexion-perfecting effect on healthy multi-ethnic skin.

Preferentially, the cosmetic benefits of this cosmetic active substance comprising at least one galactomannan-enriched hydrolysate obtained from Medicago sativa seeds, are said to be mixed and multi-ethnic. The invention is therefore preferentially aimed at the cosmetic use of this active substance for healthy Caucasian, Asian, African-American and Hispanic-American skin. In fact, the inventor observed in the course of their work that such an active substance could meet the needs of mixed and multi-ethnic beauty.

More preferentially, the invention is aimed at the cosmetic use of this active substance for its complexion-perfecting, moisturizing and anti-aging effects.

To achieve this, this active substance comprising at least one galactomannan-enriched hydrolysate obtained from Medicago sativa seeds, is advantageously a hydrolysate comprising at least 60% galactomannans by weight of the total weight of sugars in the hydrolysate.

According to a variant, the active substance according to any of the objects previously described, or the composition comprising it, is intended for use on all types of healthy skin, without discriminating by ethnicity, gender or age. In such a case, that substance has a complexion-perfecting effect, preferentially complexion-perfecting, moisturizing and anti-aging effects on all types of healthy skin. Thus, the invention also relates to the cosmetic use of the active substance according to the invention comprising at least one extract obtained from Medicago sativa seeds, said extract comprising at least 35% sugars and at most 25% proteins, for its complexion-perfecting effect on all types of healthy skin, preferentially its combined complexion-perfecting, moisturizing and anti-aging effects.

The active substance according to the invention thus makes it possible to meet the needs of so-called mixed and multi-ethnic beauty. Indeed, the active substance according to the invention or the composition according to the invention acts preferentially on all skin types, in particular Caucasian, Asian, African-American, and Hispanic-American skin.

Thus, according to an object of particular interest, the invention relates to the cosmetic use of the active substance according to any of the previously described embodiments or of a composition comprising it, intended for cosmetic treatment by topical application to the skin, for its complexion-perfecting effects, or its combined complexion-perfecting, moisturizing and anti-aging effects on healthy skin of the Caucasian, Asian, African-American, and Hispanic-American types.

The invention is now illustrated by non-limiting examples of compositions according to the invention and by results.

### EXAMPLES

### Example 1: Active Substance According to the Invention

The active substance of example 1 is obtained from a duckweed of the species Medicago sativa. The active substance of example 1 is obtained by the following method:
a. Solubilization of Medicago sativa seeds in water at a rate of at least 50 g/L,
b. Extraction by enzymatic hydrolysis,
c. Separation of the soluble and insoluble phases,
d. Heat treatment,
e. Concentration of the soluble phase,
f. Purification by ion chromatography,
g. Potentially, filtration and sterilizing filtration.

The active substance according to Example 1 consists of an extract of Medicago sativa with the following characteristics in particular:
Content of Dry Materials=26.9 g/L, of which:
Content of proteins=5.2 g/L, that is to say 19% by weight relative to the dry matter,
Content of minerals=9% by weight relative to the dry matter
Content of total sugars=20.1 g/L, that is to say 72% by weight relative to the dry matter,
49% of the sugars have a molecular weight between 360 and 3,600 Da and 34% of the sugars have a molecular weight between 3,600 Da and 1,260 kDa,
the sugars are made up of 45.9% galactose, 36.6% mannose, 14.4% glucose and 3.1% fructose.

The content of minerals was determined by weighing the residues from incineration at 550° C. in an electric muffle furnace, to obtain the mineral ash content. The content of proteins was determined using the LOWRY method (Lowry et al., Protein measurement with the folin reagent, J. Biol. Chem., 193, 265, 1951).

### Evaluation of the Efficacy of the Active Substance According to the Invention

### Trial 1—In Vitro Evaluation of the Efficacy of the Active Substance According to the Invention on Epidermal Functions.

The objective of this study is to evaluate the efficacy of the active substance according to the invention on epidermal functions Maintaining the epidermal barrier helps protect the skin. Several protein components are essential to establish and maintain this barrier function, namely:
Ki-67, a key marker for the proliferation of the cells of the basal layer of the epidermis.
loricrin, a protein which plays an important role in terminal differentiation.
claudin-1, a major component of the tight junctions responsible for cell cohesion.

Water homeostasis is also essential to the skin's physiological balance. Its maintenance is guaranteed by a set of fine regulations based in particular on specific biological markers such as aquaporin 3.

Finally, the dermal-epidermal junction performs several fundamental functions within the skin. In particular, it acts as a mechanical support for adhesion between the epidermis and dermis, and as a selective barrier to control molecular and cellular exchanges between these two compartments. This dermal-epidermal junction is made up of several proteins involved in maintaining its integrity, such as:
integrin b4, a hemidesmosomal transmembrane molecule and laminin receptor;
laminin 332, present in the anchoring filaments;
collagen VII, found in the anchoring fibrils.

Consequently, this study aims to evaluate these different markers. It was performed by immunohistofluorescence and staining on young and aged reconstructed skin for lipid staining, in the presence of a fluorescent probe. Immunohistofluorescence labeling was performed with anti-Ki-67, anti-loricrin, anti-claudin-1, anti-aquaporin 3, anti-integrin β4, anti-laminin 332 and anti-collagen VII antibodies, allowing the synthesis of each protein to be measured.

The results are presented in Tables 1 and 2, respectively describing the results on the physical and hydric barrier, as well as on the anchoring of the epidermis to the dermis.

TABLE 1

| Young reconstructed | | Aged reconstructed | |
|---|---|---|---|
| skin | | skin | |
| Con- | INVENTION | Con- | INVENTION |
| trol | 0.5% | trol | 0.5% |

Ki-67

| | | | | |
|---|---|---|---|---|
| Ki-67-positive cells (%) | 42 | 56 | 27 | 44 |
| Ability to increase | | +33 | | |
| synthesis (%) | | | | |
| Ability to restore | | | | +113 |
| synthesis (%) | | | | |

Loricrin

| | | | | |
|---|---|---|---|---|
| Synthesis (×10⁴ AU) | 490 | 589 | 255 | 368 |
| Ability to increase | | +20 | | |
| synthesis (%) | | | | |
| Ability to restore | | | | +48 |
| synthesis (%) | | | | |

Claudin-1

| | | | | |
|---|---|---|---|---|
| Synthesis (×10⁴ AU) | 345 | 424 | 205 | 338 |
| Ability to increase | | +23 | | |
| synthesis (%) | | | | |
| Ability to restore | | | | +95 |
| synthesis (%) | | | | |

Neutral lipids

| | | | | |
|---|---|---|---|---|
| Synthesis (×10⁵ AU) | 240 | 413 | 61 | 101 |
| Ability to increase | | +72 | | |
| synthesis (%) | | | | |
| Ability to restore | | | | +22 |
| synthesis (%) | | | | |

Aquaporin 3

| | | | | |
|---|---|---|---|---|
| Synthesis (×10⁵ AU) | 201 | 255 | 109 | 180 |
| Ability to increase | | +27 | | |
| synthesis (%) | | | | |
| Ability to restore | | | | +77 |
| synthesis (%) | | | | |

TABLE 2

| Young reconstructed | | Aged reconstructed | |
|---|---|---|---|
| skin | | skin | |
| Con- | INVENTION | Con- | INVENTION |
| trol | 0.5% | trol | 0.5% |

Integrin b4

| | | | | |
|---|---|---|---|---|
| Synthesis (×10⁴ AU) | 456 | 585 | 309 | 380 |
| Ability to increase | | +28 | | |
| synthesis (%) | | | | |
| Ability to restore | | | | +48 |
| synthesis (%) | | | | |

Laminin 332

| | | | | |
|---|---|---|---|---|
| Synthesis (×10⁵ AU) | 539 | 643 | 481 | 592 |
| Ability to increase | | +19 | | |
| synthesis (%) | | | | |
| Ability to restore | | | | +191 |
| synthesis (%) | | | | |

Collagen VII

| | | | | |
|---|---|---|---|---|
| Synthesis (×10⁴ AU) | 110 | 134 | 72 | 88 |
| Ability to increase | | +22 | | |
| synthesis (%) | | | | |
| Ability to restore | | | | +42 |
| synthesis (%) | | | | |

Tested at 0.5% on young and aged reconstructed skin, the active substance according to the invention significantly increases the synthesis of physical and water barrier proteins, as well as those involved in anchoring the epidermis to the dermis. The active substance according to the invention thus makes it possible to maintain a functional epidermal barrier.

### Trial 2—In Vitro Evaluation of the Efficacy of the Active Substance According to the Invention on Mixed and Multi-Ethnic Pro-Collagen Activity.

The objective of this study is to evaluate the ability of an active substance according to the invention to strengthen and preserve the dermal matrix.

The study was carried out by ELISA assay on human fibroblasts, with or without UVA exposure. The fibroblasts are derived from women or men, young or mature, and of different ethnicities (Caucasian, Asian, African-American).

The procedure is as follows. Human fibroblasts are seeded and incubated at 37° C., then treated with the active substance according to the invention at 0.25%, 0.50% and 1.00% (VN). The cells are then incubated at 37° C. and the supernatants recovered and stored at −80° C. pending ELISA assay. To study MMP-1 levels, the fibroblasts are irradiated with a UV lamp.

The results are presented in tables 3 to 4 below.

TABLE 3

| Collagen I synthesis | | |
|---|---|---|
| (ng/mg proteins) | | Efficacy (%) |
| | INVENTION | Invention |
| Control | 1.00% | 1.00%/Control |

Caucasian

| | | | |
|---|---|---|---|
| Young women | 354 | 1509 | +326 |
| Young men | 299 | 1127 | +277 |
| Mature women | 294 | 1231 | +319 |
| Mature men | 357 | 1786 | +400 |

Asian

| | | | |
|---|---|---|---|
| Mature women | 261 | 788 | +202 |
| Mature men | 258 | 844 | +227 |

African-American

| | | | |
|---|---|---|---|
| Young women | 294 | 711 | +142 |

TABLE 4

| MMP-1 content (ng/mg proteins) | | | Ability to limit |
|---|---|---|---|
| | Irradiated | | MMP-1 |
| Fibroblasts | fibroblasts | | synthesis (%) |
| not irradiated | | INVENTION | INVENTION |
| Control | Control | 1.0% | 1.0%/Control |

Caucasian

| | | | | |
|---|---|---|---|---|
| Young women | 10 | 99 | 63 | +40 |
| Young men | 5 | 106 | 77 | +29 |
| Mature women | 2 | 62 | 30 | +53 |
| Mature men | 13 | 69 | 45 | +43 |

Asian

| | | | | |
|---|---|---|---|---|
| Mature women | 28 | 76 | 61 | +31 |
| Mature men | 8 | 103 | 51 | +55 |

African-American

| | | | | |
|---|---|---|---|---|
| Young women | 1 | 11 | 6 | +50 |

Tested at 1% on mixed human fibroblasts of different ages and ethnicities, the active substance according to the invention significantly stimulates collagen I synthesis and significantly limits MMP-1 synthesis, irrespective of sex, age or ethnicity. Thus, the active substance according to the invention preserves and strengthens the dermal matrix, whatever the skin type.

### Trial 3—In Vivo Evaluation of the Efficacy of the Active Substance According to the Invention.

The biological activities and cosmetic benefits of the active substance according to the invention at 2.5% in an emulsion were assessed in vivo, in mixed panels of young and mature subjects from different ethnic groups.

The composition of the face study panels is detailed in Table 5 below.

TABLE 5

| | Products tested and | Number of subjects / |
|---|---|---|
| | application methods | average age |
| Young | | |
| panels | | |
| Caucasian | placebo formula | 20 healthy subjects: (2 |
| | formula containing the active | men and 18 women) |
| | substance according to the | average age 36 |
| | invention at 2.5% | |
| | 21 days of twice-daily half-face | |
| | application | |
| Hispanic- | placebo formula | Placebo group: |
| American | formula containing the active | 29 healthy subjects (9 men |
| | substance according to the | and 20 women) |
| | invention at 2.5% | average age 23 |
| | 21 days of twice-daily full-face | INVENTION group: |
| | application | 25 healthy subjects (6 men |
| | | and 19 women) |
| | | average age 23 |
| African- | formula containing the active | INVENTION group: |
| American | substance according to the | 30 healthy subjects (6 men |
| | invention at 2.5% | and 24 women) |
| | 21 days of twice-daily full-face | average age 34 |
| | application | |
| Mature | | |
| panels | | |
| Caucasian | placebo formula | Placebo group: |
| | formula containing the active | 19 healthy subjects (1 man |
| | substance according to the | and 18 women) |
| | invention in a 2.5% emulsion | average age 64 |
| | 42 days of twice-daily full-face | INVENTION group: |
| | application | 19 healthy subjects (19 |
| | | women) |
| | | average age 64 |
| Asian | placebo formula | 34 healthy subjects (34 |
| | formula containing the active | women) |
| | substance according to the | average age 55 |
| | invention at 2.5% | |
| | 42 days of twice-daily half-face | |
| | application | |

### Trial 3.1—Evaluation of Barrier Function Quality on a Young Caucasian Panel

To assess the quality of the barrier function, insensible water losses were measured using a Tewameter. The results are presented in table 6 below.

TABLE 6

Variation/D0 (%)

| | D7 | D21 |
|---|---|---|
| Placebo | 4.2 | 6.2 |
| Active substance 2.5% | −8.5 | −12.6 |
| Variation/Placebo (%) | −12.6 | −18.8 |

Starting at 7 days of twice-daily application, the active substance according to the invention, formulated at 2.5%, significantly improved the quality of the cutaneous barrier in young Caucasian volunteers by reducing water loss. This effect intensifies after 21 days of treatment. The active substance according to the invention thus encourages the strengthening of the barrier function.

### Trial 3.2—Evaluation of the Ability to Reduce Protein Oxidation on the Young Caucasian Panel

Oxidized proteins were studied by fluorescent labeling on strippings. After 7 days of twice-daily treatment with the active substance according to the invention at 2.5%, the quantity of oxidized proteins present on the skin surface of young Caucasian subjects decreased by 11.1%. This effect continues after 21 days of treatment with a 10.5% reduction in the oxidized protein rate.

By reducing the formation of oxidized proteins, the active substance according to the invention improves light transmission through the stratum corneum and thus helps improve skin radiance.

### Trial 3.3—Evaluation of Cell Renewal on a Young and Mature Caucasian Panel

Cell renewal was assessed on the inner forearms of a panel comprising 19 subjects of average age 34 (3 men and 16 women) and 17 subjects of average age 67 (1 man and 16 women). The study was based on photographs taken at regular intervals over 14 days, using a C-Cube dermoscope. The active substance according to the invention in a 2.5% emulsion and the placebo formula were applied to the inside of the forearms on a previously defined area colored with a formula containing dihydroxyacetone (DHA):
14 days prior to staining (pre-treatment phase).
14 days after staining (treatment phase).

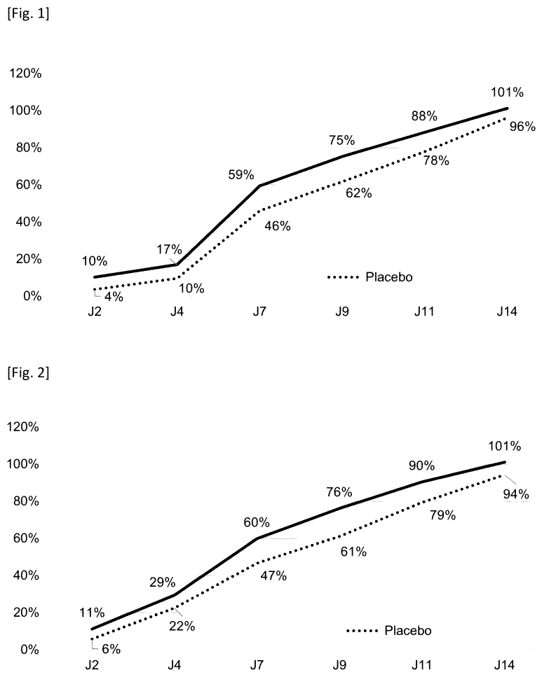

The results are presented in FIG. 1 and FIG. 2.

The 2.5% active substance in the invention stimulates epidermal renewal in both young and mature Caucasian volunteers: DHA is eliminated more rapidly after treatment with the active substance in the invention compared with the placebo formula. It therefore promotes epidermal renewal in young and mature Caucasian volunteers, significantly increasing their skin's ability to eliminate DHA as early as two days after application.

### Trial 3.4—Evaluation of Dermal Matrix Quality on a Mature Caucasian Panel

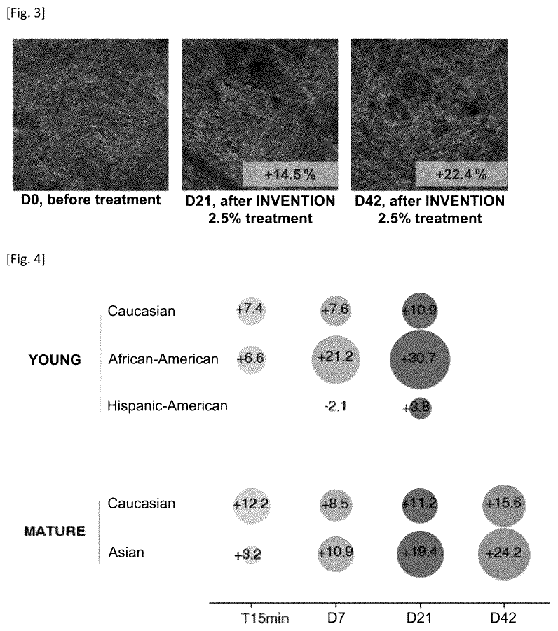

Fiber quality was studied using LC-OCT (results shown in FIG. 3), while dermal density was studied using high-frequency ultrasound.

The results of the high-frequency ultrasound are presented in table 7 below.

TABLE 7

Variation/D0 (%)

| | D21 | D42 |
|---|---|---|
| Placebo | −2.0 | −1.3 |
| Active substance 2.5% | 7.7 | 13.2 |
| Variation/Placebo (%) | 9.7 | 14.5 |

The active substance according to the invention, formulated as a 2.5% emulsion, promotes restructuring of the dermal matrix altered with age in mature Caucasian volunteers. After 21 days of treatment, compared to before treatment, the active substance according to the invention helps to lengthen dermal matrix fibers, improving the quality of dermal matrix fibers by 14.5%. Dermal density is also increased by 7.7%. These effects intensify after 42 days of treatment.

### Trial 4—In Vivo Evaluation of the Efficacy of the Active Substance According to the Invention on its Ability to Act as a Complexion Perfector

### Trial 4.1—Complexion Radiance Evaluation

Complexion radiance was studied by clinical scoring in young and mature Caucasian subjects, as well as in African-American, Asian and Hispanic-American subjects, using a Glossymeter.

The results are shown in FIG. 4.

As early as 15 minutes after a single application, a significant improvement in skin radiance was measured for Caucasian, Asian and African-American panels. Advantageously, this effect continues and intensifies after 21 days of treatment with a significant increase in the radiance parameter of the complexion observed:
among young Caucasians: +10.9%,
among young African-Americans: +30.7%,
in mature Caucasian and Asian subjects: +11.2% and +19.4% respectively.

Thus, the active substance according to the invention enhances the complexion's radiance immediately and continuously. This effect is multi-ethnic.

### Trial 4.2—Evaluation of Post-Inflammatory Hyperpigmentation on Young Hispanic-American and African-American Panels.

The complexion can be affected by the presence of spots such as hyperpigmentation marks. Reducing these pigmentary disorders helps even out skin tone.

Post-inflammatory hyperpigmentation marks were studied by image analysis from the Hispanic-American panel and by clinical scoring in African-American subjects.

The results are presented in table 8 below.

TABLE 8

Variation/D0 (%)

| | D7 | D21 |
|---|---|---|
| Hispanic-American | −17.5 | −21.7 |
| African-American | −8.9 | −16.6 |

Within 7 days, the active substance according to the invention significantly reduced the visibility of recent post-inflammatory hyperpigmentation marks: by 17.5% in Hispanic-American subjects and by 8.9% in African-American subjects. This effect is even more pronounced after 21 days of treatment, with a 21.7% reduction in pigmentary disorders in Hispanic-American subjects and a 16.6% reduction in African-American subjects.

The invention's active substance thus demonstrates a rapid anti-pigmentation effect, helping to even out skin tone. This effect is multi-ethnic.

### Trial 4.3—Study of Senescence Spots on Mature Caucasian and Asian Panels

The natural aging of the skin is a phenomenon that leads to the appearance of age spots. To maintain an even complexion, it is possible try to reduce the visibility of these spots.

Age spots were studied by image analysis from C-Cube in Caucasian subjects and by measurement using a Mexameter in Asian subjects.

The results are presented in table 9 below.

TABLE 9

Variation/D0 (%)

| | D21 | D42 |
|---|---|---|
| Caucasian | −5.7 | −7.5 |
| Asian | −18.2 | −26.6 |

As early as 21 days of treatment with the active substance according to the invention, the appearance of age spots was significantly reduced in mature Caucasian and Asian volunteers. This effect intensified after 42 days of treatment, with a 7.5% reduction in spot visibility in Caucasian subjects and a 26.6% reduction in Asian volunteers.

Thus, the active substance according to the invention also reduces the appearance of spots on mature skin, helping to maintain an even complexion. This effect is multi-ethnic.

### Trial 5—Evaluation of the Moisturizing-Smoothing Effect of the Active Substance According to the Invention

### Trial 5.1—Hydration Rate Evaluation on Young Caucasian, Hispanic-American and African-American Panels.

The study of hydration levels in young Caucasian, Hispanic-American and African-American panels was carried out using a Corneometer.

The results are presented in table 10 below.

TABLE 10

Variation/D0 (%)

| | D7 | D21 |
|---|---|---|
| Caucasian | 14.6 | 17.7 |
| Hispanic-American | 43.3 | 65.9 |
| African-American | 13.0 | 7.1 |

After 7 days of use, a significant increase in skin moisturization with the active substance according to the invention was measured. The active substance according to the invention thus has a moisturizing effect. This effect is multi-ethnic.

### Trial 5.2—Hydration Rate Evaluation on Young Caucasian, Hispanic-American and African-American Panels.

The smoothing effect was studied using fringe projection in Caucasian subjects, image analysis in Hispanic-American subjects and clinical scoring in African-American subjects.

The results are presented in table 11 below.

TABLE 11

Variation/D0 (%)

| | D7 | D21 |
|---|---|---|
| Caucasian | −6.1 | −3.9 |
| Hispanic-American | 0 | −13.2 |
| African-American | −9.5 | −26.2 |

As early as 7 days of use, the active substance according to the invention significantly promotes the smoothing of microreliefs and fine lines in young Caucasian and African-American subjects. This effect increases after 21 days of treatment in the Hispanic-American and African-American panels, with a significant reduction of 13.2% and 26.2% respectively.

The active substance according to the invention thus has a smoothing effect, helping to improve the quality of the skin's surface, and this effect is multi-ethnic.

### Trial 5.3—Evaluation of Skin Texture on Young Caucasian and African-American Panels

Skin grain appearance was studied by clinical scoring in Caucasian and African-American subjects, and by image analysis in Hispanic-American subjects.

The results are presented in table 12 below.

TABLE 12

Variation/D0 (%)

| | D7 | D21 |
|---|---|---|
| Caucasian | −12.7 | −14.3 |
| Hispanic-American | −10.6 | −13.1 |
| African-American | −15.7 | −21.7 |

As early as 7 days, the active substance according to the invention significantly reduced the visibility of skin pores in Caucasian subjects by 12.7% and in African-American subjects by 15.7%, and tended to refine pores in Hispanic-American subjects. This effect increases after 21 days of treatment, with a significant reduction in pore visibility of 14.3% in Caucasian volunteers, and 21.7% in African-American subjects. A 13.1% reduction was also observed in Hispanic-American subjects.

Thus, by reducing the visibility of pores, the active substance according to the invention refines skin texture and helps to improve the aesthetic inconveniences associated with the problems of young skin in different populations.

### Trial 6—Evaluation of the Anti-Aging Effect of the Active Substance According to the Invention

### Trial 6.1—Evaluation of the Anti-Wrinkle Effect on Mature Caucasian and Asian Panels

The anti-wrinkle effect was assessed by fringe projection on mature Caucasian and Asian subjects.

The results are presented in table 13 below.

TABLE 13

| | Variation/D0 (%) | | |
|---|---|---|---|
| | D7 | D21 | D42 |
| Caucasian | −7.9 | −10.8 | −8.5 |
| Asian | −11.7 | −11.3 | −17.8 |

The active substance according to the invention, formulated at 2.5%, has an anti-wrinkle effect on mature volunteers as early as 7 days of use, with a significant reduction in wrinkles of 7.9% in Caucasian volunteers and 11.7% in Asian volunteers. This effect continued after 21 and 42 days for both panels. Thus, the active substance according to the invention offers a rapid, long-lasting anti-wrinkle effect, whatever the skin type.

### Trial 6.2—Study of Firmness on Mature Caucasian and Asian Panels

Skin firmness was assessed using a Cutometer in mature Caucasian and Asian subjects.

The results are presented in table 14 below.

TABLE 14

Variation/D0 (%)

| | D21 | D42 |
|---|---|---|
| Caucasian | 12.7 | 15.7 |
| Asian | 4.1 | 10.8 |

As early as 21 days of treatment, the active substance according to the invention formulated at 2.5% significantly improves the firmness of the skin of Caucasian volunteers by 12.7%. This effect intensified after 42 days of treatment, with a significant increase in firmness of 15.7% in Caucasian volunteers and 10.8% in Asian volunteers. Thus, by improving the firmness of mature skin, whatever the skin type, the active substance according to the invention offers powerful anti-aging power.

### Example 2: Active Substance Outside the Invention

The product in Example 2 is obtained from an extract of the Ceratonia siliqua species known for its high galactomannan content. The product of example 2 is obtained by the following method:
a. Solubilization of Ceratonia siliqua seeds in water at a rate of at least 50 g/L,
b. Extraction by enzymatic hydrolysis,
c. Separation of the soluble and insoluble phases,
d. Heat treatment,
e. Concentration of the soluble phase.

The product according to example 3 consists of an extract of Ceratonia siliqua seeds with the following characteristics:
Content of Dry Materials=61.4 g/L, of which:
Protein content (Lowry assay)=7.2 g/L or 12% by weight of dry matter,
Content of total sugars=52 g/L, that is to say 85% by weight relative to the dry matter,
100% have a molecular weight between 360 Da and 100 kDa.

Efficacy results, namely collagen I synthesis on human fibroblasts, are presented in Table 15 below.

TABLE 15

| | Synthesis of | |
|---|---|---|
| | collagen I | Efficacy |
| | (ng/mg proteins) | (%) |
| Control | 344 | |
| INVENTION - Example 1 at 0.5% | 1005 | +192 |
| INVENTION - Example 1 at 1.0% | 1425 | +314 |
| NOT IN INVENTION - Example 2 | 297 | — |
| at 0.5% | | |
| NOT IN INVENTION - Example 2 | 317 | — |
| at 1.0% | | |

This active substance, obtained by a method similar to the invention, but from a different raw material, shows no efficacy on collagen I synthesis. Thus, the activity of the active substance according to the invention is linked to the size and structure of the specific galactomannans from Medicago sativa.

### Example 3: Composition According to the Invention

An example of a formulation comprising the active substance according to the invention in the form of anti-aging serum is presented in table 16 below.

TABLE 16

| | Ingredients | % |
|---|---|---|
| A1 | Purified Water | q.s. 100 |
| | Preservative | qs |
| | Erythritol | 3.00 |
| A2 | Sclerotium Gum & Xanthan Gum | 0.30 |
| B | Steareth-2 | 1.84 |
| | Steareth-21 | 1.16 |
| | Caprylyl Methicone | 5.00 |
| | Dimethicone | 10.00 |
| C | Dimethicone & Dimethicone/Vinyl Dimethicone | 6.00 |
| | Crosspolymer | |
| D | Polyacrylate-13 & Polyisobutene & Polysorbate 20 | 0.50 |
| E | Active substance, Invention | 2.50 |

The composition of example 3 can in particular be obtained by the following method:
Add A2 to A1 with stirring and heat to 70° C.
Place B with stirring and heat to 70° C.
Emulsify B in A under shearing stirring for 10 minutes.
At 60° C., add C under shearing agitation. Stir for 5 minutes.
At room temperature, with gentle stirring, add D, then E.

The composition then takes the form of a fluid, glossy, off-white emulsion.

### Example 4: Composition According to the Invention

An example of a formulation comprising the active substance according to the invention in the form of a firming cream is presented in table 17 below.

TABLE 17

| | Ingredients | % |
|---|---|---|
| A1 | Purified Water | q.s. 100 |
| | Preservative | qs |
| | Butylene Glycol | 4.00 |
| A2 | Glycerin | 2.00 |
| | Sclerotium Gum & Xanthan Gum | 0.30 |
| B | Cetearyl Alcohol & Cetearyl Glucoside | 2.00 |
| | Coco-Glucoside & Coconut Alcohol | 2.00 |
| | C10-18 Triglycerides | 1.00 |
| | Oleyl Erucate | 5.00 |
| | Isocetyl Stearate | 4.00 |
| | Dicaprylyl Carbonate | 6.00 |
| | Propanediol Dicaprylate | 4.00 |
| | Dimethicone | 2.00 |
| | Acrylates/C10-30 Alkyl Acrylate Crosspolymer | 0.30 |
| C | Active substance, Invention | 2.50 |
| D | Soda Solution 28% | qs pH |

The composition of example 4 can in particular be obtained by the following method:
Add A2 to A1 with stirring and heat to 80° C.
Place B with stirring and heat to 80° C.
Emulsify B in A under shearing stirring for 10 minutes.
At room temperature, with gentle stirring, add C.
Adjust pH to 5.0-5.5 with D.

The composition then takes the form of a very thick, off-white emulsion.

### Example 5: Composition According to the Invention

An example of a formulation comprising the active substance according to the invention in the form of a smoothing gel cream is presented in table 18 below.

TABLE 18

| | Ingredients | % |
|---|---|---|
| A | Purified Water | q.s. 100 |
| | Butylene Glycol | 3.00 |
| | Glycerin | 2.00 |
| | Propylene glycol | 2.00 |
| B | Acrylates/C10-30 Alkyl Acrylate Crosspolymer | 0.40 |
| C | Caprylic/Cupric/Succinic Triglyceride | 4.00 |
| | Dicaprylyl Carbonate | 4.00 |
| | Octyldodecanol | 4.00 |
| | Ricinus Communis (Castor) Seed Oil | 2.00 |
| | Tocopherol | 0.01 |
| D | Soda Solution 28% | qs pH |
| E | Preservative | qs |
| F | Active substance, Invention | 2.50 |
| G | Silica | 2.00 |

The composition of example 5 can in particular be obtained by the following method:
Disperse B in A under moderate stirring.
Add C to AB under vigorous agitation.
Adjust the pH with J to pH 5.0-5.5.
Keep stirring for 10 minutes.
Under moderate stirring, add E, F, and G.

The composition then takes the form of a glossy, off-white gel cream.

### Example 6: Composition According to the Invention

An example of a formulation comprising the active substance according to the invention in the form of an anti-wrinkle foundation is presented in table 19 below.

TABLE 19

| | Ingredients | % |
|---|---|---|
| A | Tri(Polyglyceryl-3/Lauryl) Hydrogenated Trilinoleate | 3.00 |
| | Cera Alba (Beeswax) | 0.40 |
| | Magnesium Stearate | 0.25 |
| | C12-15 Alkyl Benzoate & Stearalkonium Hectorite & | 7.00 |
| | Propylene Carbonate | |
| | Diisopropyl Sebacate | 13.00 |
| B | Crambe Abyssinica Seed Oil | 7.00 |
| | CI 77499 (Iron Oxides) & Isostearyl Sebacate & | 0.15 |
| | Disodium Stearoyl Glutamate & Aluminum Hydroxide | |
| | CI 77491 (Iron Oxides) & Isostearyl Sebacate & | 0.30 |
| | Disodium Stearoyl Glutamate & Aluminum Hydroxide | |
| | CI 77891 (Titanium Dioxide) & Isostearyl Sebacate & | 7.00 |
| | Disodium Stearoyl Glutamate & Aluminum Hydroxide | |
| | CI 77492 (Iron Oxides) & Isostearyl Sebacate & | 0.70 |
| | Disodium Stearoyl Glutamate& Aluminum Hydroxide | |
| C | Purified Water | q.s. 100 |
| | Preservative | qs |
| | Glycerin | 4.00 |
| | Magnesium Sulfate | 1.00 |
| D | Kaolin | 2.50 |
| E | Active substance, Invention | 2.50 |

The composition of example 6 can in particular be obtained by the following method:
Heat A to 80° C. and homogenize with vigorous stirring.
Grind B in the triple-roller and transfer to A. Stir until homogenized.
Set B to stir and heat to 80° C.
Add C very slowly to AB with vigorous stirring.
Homogenize for 10 min with shearing stirring.
At 30° C., add D then E and homogenize for 1 min under shearing stirring.

The composition then takes the form of a thick, shiny, light-beige emulsion.

1. A cosmetic use of an active substance comprising galactomannans derived from a hydrolysate obtained from Medicago sativa seeds, for its complexion-perfecting effect on healthy skin.

2. The cosmetic use of an active substance according to claim 1, characterized in that the healthy skin is Caucasian and Asian and African-American and Hispanic-American healthy skin.

3. The cosmetic use of an active substance according to claim 2, characterized in that it has complexion-perfecting, moisturizing and anti-aging effects.

4. The cosmetic use of an active substance according to claim 1, characterized in that the sugar fraction of the hydrolysate comprises at least 60% galactomannans by weight of the total weight of sugars in the extract.

5. A cosmetic active substance comprising at least one extract obtained from the seeds of Medicago sativa, characterized in that the Medicago sativa extract comprises, by weight of the total weight of the extract, at least 35% sugars and at most 25% proteins.

6. The cosmetic use of an active substance according to claim 5, characterized in that the sugar fraction of the extract comprises at least 60% galactomannans by weight of the total weight of sugars in the extract.

7. The cosmetic active substance according to claim 5, characterized in that the sugar fraction of the extract comprises at least 60% galactomannans with molecular weights of between 360 Da (DP2) and 1260 kDa (DP7000), by weight of the total weight of sugars in the extract.

8. The cosmetic active substance according to claim 5, characterized in that the sugar fraction of the extract comprises at least 30% galactomannans with molecular weights of between 3.6 Da (DP20) and 1260 kDa (DP7000), by weight of the total weight of sugars in the extract.

9. The cosmetic active substance according to claim 5, characterized in that the extract of Medicago sativa is a is a hydrolysate.

10. The active substance for its use according to claim 9, characterized in that the hydrolysate of Medicago sativa is an enzymatic hydrolysate.

11. The cosmetic active substance according to claim 5, characterized in that the extract is capable of being obtained by a method comprising the following steps:
a. Solubilization of Medicago sativa seeds in water at a rate of at least 50 g/L,
b. Extraction by hydrolysis, preferentially enzymatic hydrolysis,
c. Separation of the soluble and insoluble phases,
d. Heat treatment,
e. Concentration of the soluble phase,
f. Purification by ion chromatography,
g. Potentially, filtration and sterilizing filtration.

12. A cosmetic composition comprising at least 0.1% by weight of an extract according to claim 5, based on the total weight of the composition.
